(19) United States
(12) Patent Application Publication
(10) Pub. No.: US 2023/0037070 A1
(43) Pub. Date: Feb. 2, 2023

(54) MULTILAYER MATTER-LIGHT DISINFECTOR

(71) Applicant: BOLB INC., Livermore, CA (US)

(72) Inventors: JIANPING ZHANG, Livermore, CA (US); Alexander Lunev, Livermore, CA (US); LING ZHOU, Livermore, CA (US); YING GAO, Livermore, CA (US)

(21) Appl. No.: 17/387,526

(22) Filed: Jul. 28, 2021

(51) Int. Cl. A61L 9/20

(52) U.S. Cl. CPC A61L 9/20; A61L 2209/12; A61L 2209/14

(57) ABSTRACT

A fluid disinfector includes one or more disinfection units connected in series along a flow path of a fluid to be disinfected so that the fluid to be disinfected can only enter one disinfection unit after exiting from an adjacent preceding disinfection unit. The disinfection unit includes a matter layer, a light layer and a germicidal light source for generating germicidal light. The matter layer is made of porous material having a first surface to receive the fluid to be disinfected and to intercept and capture pathogens from the fluid to be disinfected; the light layer is a space permeable to the fluid and transparent to the germicidal light. When in operation, the light layer contains the germicidal light impinging on the first surface of the matter layer.

### TECHNICAL FIELD

The present disclosure relates to a fluid disinfector using disinfection light and porous material to disinfect fluid.

### DESCRIPTION OF THE RELATED ART

More and more evidences reveal that airborne viruses are responsible for epidemic/pandemic outbreaks. Air disinfection, therefore, especially for air in confined spaces such as indoors or in transport vehicles, is foreseen to be mandatory in future to eliminate pandemic outbreaks such as influenza and Covid-19. Air disinfection, unlike surface and water disinfection, has its own characteristics. The most vital one is that commercially viable air disinfection technologies need to be able to treat air of large flow rates, for example, from hundreds of liters per minute (LPM) for vehicles, to thousands LPM for small rooms, to tens even hundreds thousand LPM for homes and public indoor spaces. Equally important is that these air disinfectors need to be germicidal efficient and cost-effective.

Filtration is a likely approach to satisfy the above two requirements for air disinfection, as filters can remove particles and pathogens from air. According to American Society of Heating, Refrigerating and Air-Conditioning Engineers (ASHRAE), air filters can have vastly different filtration efficiency, depending on their Minimum Efficiency Reporting Value (MERV). The MERV ratings, from 1 to 20, give an idea of how well filters can filter out 0.3-to-10-micron particles. For example, A MERV 10 filter removes 50%-64.9% of air pollutants with an average particle size between 1 and 3 microns, and more than 85% of air pollutants with an average particle size between 3 and 10 microns or greater. A MERV 14 filter removes 75%-84% of air pollutants with an average particle size between 0.3 and 1 microns and more than 90% of air pollutants with an average particle size between 1 and 3 microns or greater.

The problem with filtration is that pathogens are only separated from air streamlines temporarily without being inactivated. These pathogens will multiply on the filters as they feed on the trapped organic particles there such as cooking oil, pollens, et al. As the pathogen population grows the pathogen desorption probability increases which will result in pollution of the room air for a short lifetime of the filters.

Meanwhile, germicidal ultraviolet light (GUV) mercury lamps have been used in hospitals for air disinfection. Recently, solid-state GUV light sources such as AlGaN based ultraviolet C-band (UVC) light-emitting diodes (LEDs) have been emerging as favorable substitute for mercury lamps, as UVC LEDs can emit light with much higher intensity, and are more adapted to various applications because of their small footprint.

Under diluted conditions, i.e., pathogens incapable of shadowing each other from GUV light, pathogen population will decay exponentially with the experienced GUV dose:

$$\frac{[ n ]}{[ {n}_{0} ]} = 1 {0}^{- \frac{J}{D}} \qquad ( 1 )$$

where [n₀], [n] are pathogen's initial and current counts or concentrations, respectively, J is GUV dose, and D is the GUV dose for pathogen of 10% survival rate. From eq. (1) it is clear that to have 10%, 1%, 0.1%, 0.01%, and 0.001% survival rates (i.e., to have kill rates of 90%, 99%, 99.9%, 99.99%, and 99.999%, respectively), one has to deliver GUV doses of D, 2D, 3D, 4D, and SD, respectively. As GUV dose is the product of GUV light intensity and exposure time, one can perfect disinfect effectiveness via increasing either GUV light intensity, or exposure time, or both.

The present disclosure discloses efficient fluid disinfectors taking the advantages of filtration and light germicidal effect.

### SUMMARY

A fluid disinfector according to an aspect of the present disclosure includes:

one or more disinfection units connected in series along a flow path of a fluid to be disinfected so that the fluid to be disinfected can only enter one disinfection unit after exiting from an adjacent preceding disinfection unit, the disinfection unit comprising a matter layer, a light layer and a germicidal light source for generating germicidal light, wherein:

the matter layer is made of porous material having a first surface to receive the fluid to be disinfected and to intercept and capture pathogens from the fluid to be disinfected;

the light layer is a space permeable to the fluid and transparent to the germicidal light, when in operation, the light layer contains the germicidal light impinging on the first surface of the matter layer.

A fluid disinfector according to another aspect of the present disclosure includes n disinfection units connected in series such that the fluid disinfector has an effective flow disinfection efficiency ρ_{eff} satisfying:

ρ_{eff}=1−(1−ρ)ⁿ

where ρ is flow disinfection efficiency of a disinfection unit,

$$ρ = 1 - \frac{[ {n}_{o u t} ]}{[ {n}_{i n} ]} , [ {n}_{in} ] , [ {n}_{out} ]$$

are pathogen concentrations in the fluid just before and after the disinfection unit, respectively, and n is a positive integer no less than 1, wherein:

the disinfection unit comprising a matter layer, a light layer and a germicidal light source for generating germicidal light, and,

the matter layer is made of porous material having a first surface to receive the fluid and to intercept and capture pathogens from the fluid;

the light layer is a space permeable to the fluid and transparent to the germicidal light, when in operation, the light layer contains the germicidal light impinging on the first surface of the matter layer.

A fluid disinfector according to still another aspect of the present disclosure includes:

one or more cylindrical disinfection units with different diameters co-axially connected in series along a flow path of a fluid to be disinfected so that the fluid to be disinfected can only enter one disinfection unit after exiting from an adjacent preceding disinfection unit, the disinfection unit comprising a matter layer, a light layer, and a germicidal light source for generating germicidal light, wherein:

the matter layer has a cylindrical shape and is made of porous material having a first surface to intercept and capture pathogens from the fluid to be disinfected, and a second surface opposite to the first surface;

the light layer is defined between the first surface of the matter layer of a disinfection unit and the second surface of the matter layer of an adjacent disinfection unit;

when in operation, the germicidal light in the light layer impinges onto the first surface of the matter layer.

### BRIEF DESCRIPTION OF THE DRAWINGS

The accompanying drawings, which are included to provide a further understanding of the disclosure and constitute a part of this application, illustrate embodiments of the invention and together with the description serve to explain the principle of the disclosure. Like reference numbers in the figures refer to like elements throughout, and a layer can refer to a group of layers associated with the same function.

FIG. 1 shows a cross-sectional illustration of a multilayer matter-light disinfector according to the present disclosure.

FIG. 2 plots the effective flow disinfection efficiency ρ_{eff} as function of the series connection number n and flow disinfection efficiency ρ of the disinfection unit for a multilayer matter-light disinfector according to the present disclosure.

FIG. 3 plots air disinfection rate in a 30 m³ room using air flow rate 3000 LPM by various kinds of multilayer matter-light disinfector.

FIG. 4 plots air disinfection rate in a 30 m³ room of different patient occupancy using air flow rate 3000 LPM by a multilayer matter-light disinfector of effective flow disinfection efficiency of 99.96%.

FIG. 5A shows a perspective view of a matter-light dual-layer as building block for a multilayer matter-light disinfector according to one aspect of the present disclosure.

FIG. 5B illustrates a cross-sectional schematic view of the matter-light dual-layer shown in FIG. 5A along the AA′ cut.

FIG. 5C shows a cross-sectional illustration of a multilayer matter-light disinfector made via series connecting a few matter-light dual-layers shown in FIGS. 5A and 5B according to the present disclosure.

FIG. 6A shows a perspective view of a matter-light dual-layer as building block for a multilayer matter-light disinfector according to another aspect of the present disclosure.

FIG. 6B illustrates a birds'-eye view of the matter-light dual-layer shown in FIG. 6A.

FIG. 6C shows a perspective view of a multilayer matter-light disinfector made via series connecting a few matter-light dual-layers shown in FIGS. 6A and 6B according to the present disclosure.

FIG. 6D illustrates a cross-sectional schematic view of the multilayer matter-light disinfector shown in FIG. 6C along the AA′ cut.

### DETAILED DESCRIPTION OF EMBODIMENTS

In the following description, for the purposes of explanation, specific details are set forth in order to provide an understanding of the disclosure. It will be apparent, however, to one skilled in the art that the disclosure can be practiced without these details. One skilled in the art will recognize that embodiments of the present disclosure, described below, may be performed in a variety of ways and using a variety of means. Those skilled in the art will also recognize additional modifications, applications, and embodiments are within the scope thereof, as are additional fields in which the disclosure may provide utility. Accordingly, the embodiments described below are illustrative of specific embodiments of the disclosure and are meant to avoid obscuring the disclosure.

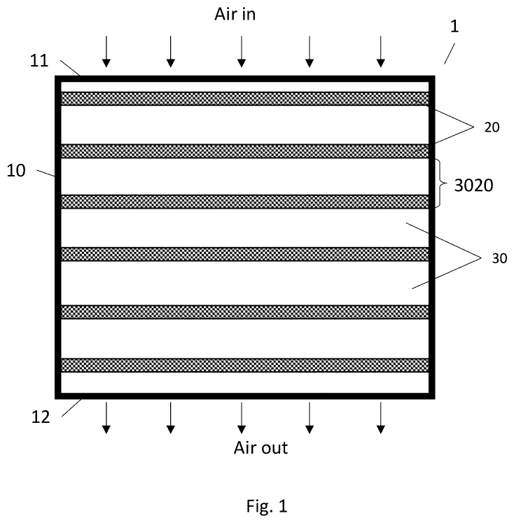

FIG. 1 shows a cross-sectional illustration of a fluid disinfector 1 according to the present disclosure. Disinfector 1, called as multilayer matter-light (MLML) disinfector in the following specification, has at least one matter layer 20 made of porous matter and one light layer 30 filled with light when in use, each playing a different role in disinfecting pathogens in fluids. The MLML disinfector 1 in general prefers to have multiple matter layers 20 alternatively arranged with light layers 30 to have improved flow disinfection efficiency. In principle, matter layer 20 is mainly made of porous materials with surface adhesion for microbes, utilizing their large surface area to intercept and capture pathogens from fluid streamlines. Matter layer 20 may also contain a mechanical support structure for housing the porous material. In some embodiments, matter layer 20 is made of porous material enclosed by a metal mesh housing and supported by a metal frame to reinforce the perimeter. For example, matter layer 20 can be a filter with suitable MERV ratings (i.e., any MERV rating in the range of 1-20, for example, of MERV rating of 6-14), selected from such as cellulose filters, polycarbonate filters, gelatin filters, polytetrafluoroethylene (PTFE) filters, activated carbon fiber filters, (granular) activated carbon filters, silica gel filters, activated alumina filters, porous or aluminum mesh filters, and synthetic zeolites filters, et al. Activated carbon fiber (ACF) filter is especially suitable for matter layer 20 because of its light weight, large surface area, superior adsorption capacity and fabric form. Generally, activated carbon fiber can be achieved via thermal treatment (400-900° C.) of organic materials such as polyester, nylon, acrylic, cotton, silk, banana peels, almond shells, coconut shells, peach stones, et al, under hot steam of water vapor, nitrogen, carbon dioxide et al. Hot steams containing water vapor can consume part of the materials and open up micro- and nano-pores to form activated carbon fiber of extremely large surface area (1000-3000 m²/g), which is essential for superior adsorption capacity when used as a gas or fluid filter. Light layer 30 is a space permeable to fluids and transparent to germicidal light, when in use, filled with germicidal light (GL). GL in light layer 30 can be introduced by any suitable means, of any suitable wavelengths. For example, GL can be introduced to the light layer 30 using a lamp, an LED, a light emitting surface, a light guiding fiber, et al. The wavelength of GL can be in the ultraviolet regime, such as 10-420 nm, which is effective in disrupting pathogens' replica materials or destroying pathogens' proteins, or in the infrared regime or microwave regime, which can heat matter layer 20 hence kill pathogens captured therein. GL in light layer 30 preferably impinges onto the surface of matter layer 20 along the fluid flowing direction. The fundamental function of matter layer 20 is to entrap pathogens and enlarge pathogens' residual time on the surface of matter layer 20, so that GL in light layer 30 impinging on the surface of matter layer 20 can achieve large action (exposure) time hence germicidal dose for the pathogens entrapped there.

When the germicidal light in light layer 30 is ultraviolet light (i.e., wavelength in the range of 10-420 nm), matter layer 20 is optionally made of porous materials of good ultraviolet reflectance, such as polytetrafluoroethylene (PTFE), porous aluminum or aluminum mesh, et al. The ultraviolet reflectance R in theory can enhance the ultraviolet light intensity within the pores of matter layer 20 by a factor of

$$\frac{1}{1 - R} .$$

Hence, if R is in 0.5-0.99, the ultraviolet intensity in the pores could be enhanced by a factor of 2-10, as a result of infinite times of reflections taking place within the pores.

The thicknesses of matter layer 20 and light layer 30 measured along the fluid flow direction can be in the range of 1-10 mm and 1-100 cm such as 10-40 cm, respectively, optimally determined by a balance of flow resistance and pathogen capture efficiency and uniform illumination of GL, respectively. The lateral dimension can be application dependent. For example, it depends on the fluid flow rate. In general, the lateral dimension can be 10-100 cm, or larger.

MLML disinfector 1 shown in FIG. 1 also include a housing 10, to provide mechanical support for matter layers 20 and confine GL and flowing fluid to be disinfected, a fluid inlet 11 and an outlet 12. Light layer 30 therefore is confined as a space sandwiched by two matter layers 20 and surrounded by housing 10. Housing 10 can be made of stainless steel, aluminum alloys, et al, allowing no fluid penetration through its sidewalls. Matter layer 20 can be fixed to housing 10 in any suitable manner. For example, one can fasten (via any common means such as screws, clamps, et al) the frame of matter layer 20 to housing 10.

In MLML disinfector 1 shown in FIG. 1, light layer 30, matter layer 20 and a portion of housing 10 form a disinfection unit 3020, and a MLML disinfector may contain more than one disinfection unit 3020 connected in series. Disinfection units 3020 connected in series in this specification means that these disinfection units are all connected in sequence along the flow path of the fluid, where fluid flow can only enter one disinfection unit after exiting its preceding disinfection unit. For example, MLML disinfector 1 shown in FIG. 1 contains 5 disinfection units 3020, all connected in series.

In MLML disinfector 1 shown in FIG. 1, disinfection units 3020 are arranged along a straight line, but they can be arranged in other configurations, such as zig-zag shape or any other suitable shape. Matter layers 20 and light layers 30 in MLML disinfector 1 may have the same or different dimension (such as thickness), material, flow disinfection efficiency and type of light source.

Furthermore, disinfection unit can be of a cylindrical shape and disinfection units of the same or different diameters can be arranged co-axially in series connection.

The operation principle of MLML disinfector can be understood as follows. Suppose that through bioburden tests, one can obtain a flow disinfection efficiency ρ (which is dependent on flow, pathogen type, et al) of disinfection unit 3020, and ρ is defined as

$$ρ = 1 - \frac{[ {n}_{o u t} ]}{[ {n}_{i n} ]} ,$$

where [nᵢₙ], [nₒᵤₜ] are pathogen concentrations (or counts) in the flowing fluid just before and after the disinfection unit, respectively. Then consider the effective flow disinfection efficiency ρ_{eff} of a MLML disinfector having n disinfection units connected in series. Suppose that this MLML disinfector is in a room of volume V for disinfecting air with flow rate G, and the pathogen counts in the room is N (initial counts N₀), then in an infinitesimal time period dt, the pathogen number change due to the MLML disinfector is dN:

$$d N = - ρ G \frac{N}{V} d t - ρ G \frac{N}{V} ( 1 - ρ ) d t - ρ G \frac{N}{V} {( 1 - ρ )}^{2} dt - … - ρG \frac{N}{V} {( 1 - ρ )}^{n - 1} d t$$

This gives,

$$N = {N}_{0} {e}^{- {ρ}_{eff} \frac{G}{V} t} \qquad ( 2 )$$

$${ρ}_{eff} = 1 - {( 1 - ρ )}^{n} \qquad ( 3 )$$

As seen from eq. (2), if without pathogen generation (i.e., no presence of sick persons), the pathogen counts in the room will decay exponentially due to disinfection by the MLML disinfector, whose effective flow disinfection efficiency ρ_{eff} is given by eq. (3).

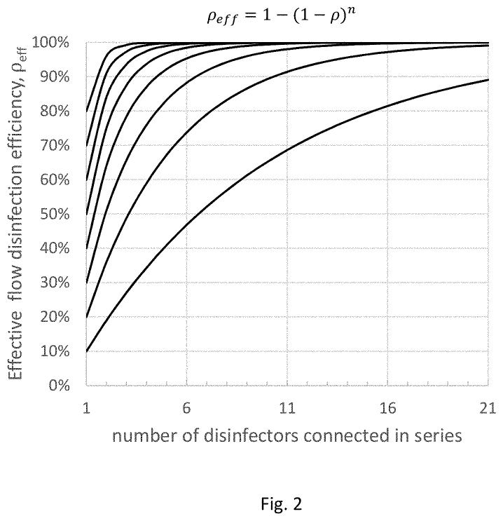

From eq. (3), the effective flow disinfection efficiency ρ_{eff} for MLML disinfector can be very large (approaching 1) as the number n of the series connected disinfection units increases. This is shown graphically in FIG. 2, which plots the effective flow disinfection efficiency ρ_{eff} as function of the series connection number n and flow disinfection efficiency ρ of the disinfection unit. As seen, for a disinfection unit of flow disinfection efficiency ρ=10%, one needs to have 21 such disinfection units connected in series to obtain an effective flow disinfection efficiency ρ_{eff} about 90%, wherein if ρ=30% and 50%, one needs to have 6 and 4 such disinfection units, connected in series to obtain an effective flow disinfection efficiency ρ_{eff} about 90%, respectively. Therefore, one aspect of the present disclosure teaches a method to make a fluid disinfector of effective flow disinfection efficiency ρ_{eff} via series connecting n disinfection units of flow disinfection efficiency ρ, to obtain ρ_{eff}=1−(1−ρ)ⁿ according to eq. 3. As seen, the larger the flow disinfection efficiency ρ, the less the series connected disinfection units needed to achieve a high effective flow disinfection efficiency ρ_{eff}.

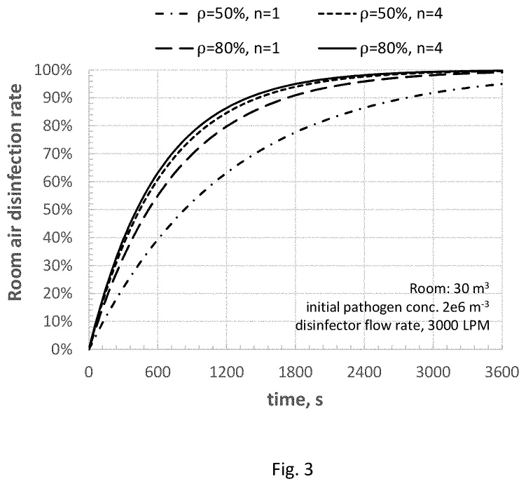

Also, fluid (such as room air) disinfection rate (or efficiency) r can be defined as

$$r = 1 - \frac{N}{{N}_{0}} ,$$

where N and N₀ are the pathogen's counts and pathogen's initial counts in the room, respectively. FIG. 3 plots disinfection rates r's for air in a 30 m³ room of initial pathogen concentration 2×10⁶ m⁻³ (without additional pathogen generation) using air flow rate 3000 LPM by four MLML disinfectors with different disinfection unit configurations: i.e., MLML disinfectors with n=1, ρ=50%; n=1, ρ=80%; n=4, ρ=50%; and n=4, ρ=80%. The observation is that for the MLML disinfectors of large series connection number n (e.g., n=4), the (room air) disinfection rate converges in a short period of time regardless of the obvious difference (i.e., ρ=50% vs 80%) in the flow disinfection efficiency ρ of the building block disinfection unit of the MLML disinfectors. FIG. 3 reveals that when 4 disinfection units connected in series, no matter the disinfection unit is of ρ=50% or ρ=80%, the respective MLML disinfectors both achieve about 99% disinfection rate in one hour for air in the 30 m³ room.

When there is pathogen generation in the room, such as patient occupancy, we have,

$$dN = - \frac{GN}{V} {ρ}_{eff} dt + mgbdt ,$$

where m is the patient number, g the breath rate, b the pathogen concentration in breath. Then,

$$N = {N}_{0} {e}^{- {ρ}_{eff} \frac{G}{V} t} + \frac{mgbV}{G {ρ}_{eff}} ( 1 - {e}^{- {ρ}_{eff} \frac{G}{V} t} ) \qquad ( 4 )$$

This mean that the presence of patient will impact the room air disinfection rate as

$$r = 1 - \frac{N}{{N}_{0}} .$$

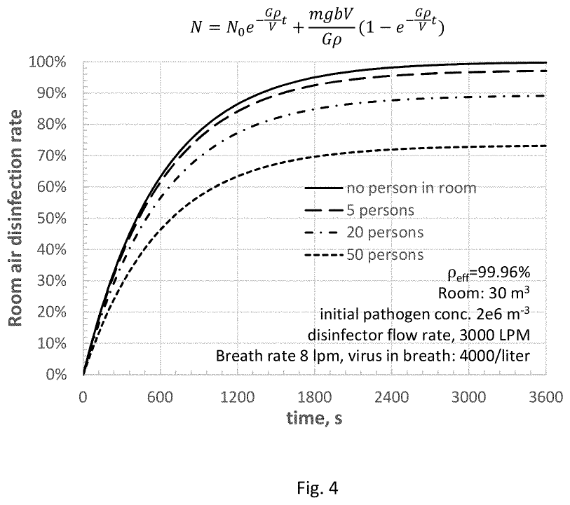

FIG. 4 plots air disinfection rates in a 30 m³ room of different patient occupancy using air flow rate 3000 LPM by a MLML disinfector of effective flow disinfection efficiency ρ_{eff}=99.96%, assuming patient breath rate is 8 LPM and pathogen concentration in breath is 4000/liter. As seen, for normal occupancy, such as 5 patients in the room, it only slightly reduces the air disinfection rate r (r from 99.75% down to 97.1% in one hour). As patient occupancy goes beyond normal, such as 20 or 50 patients in the 30 m³ room, the air disinfection rater would drop (from 99.75%) to 89.11% and 73.14% in one hour, respectively.

It is also noted that if such n disinfection units are connected in parallel to form a fluid disinfector, the effective flow disinfection efficiency would still be ρ, but the pathogen's counts would decay according to equation

$$N = {N}_{0} {e}^{- ρ \frac{n G}{V} t} + \frac{mgbV}{nG ρ} ( 1 - {e}^{- ρ \frac{n G}{V} t} ) ,$$

with the total flow rate being nG instead of G.

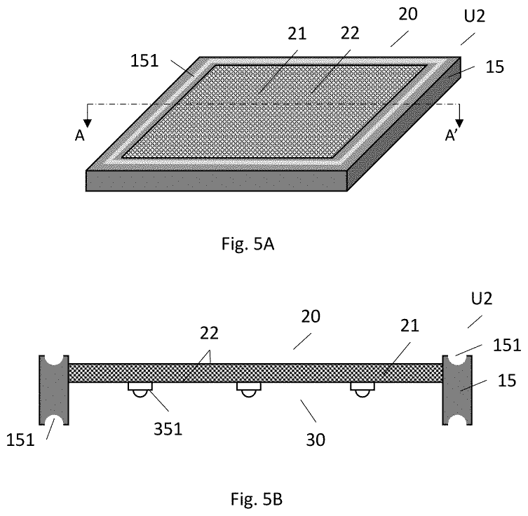

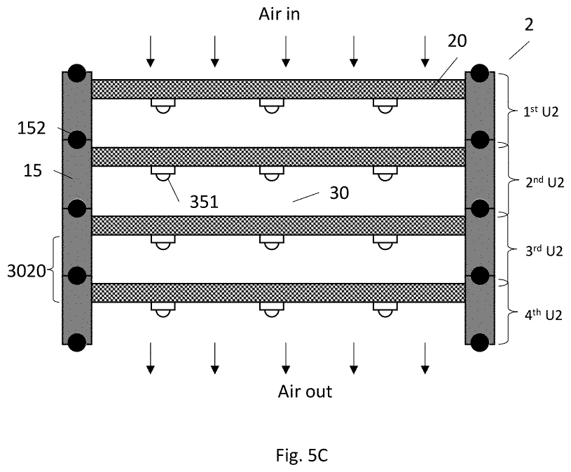

FIG. 5A shows a perspective view of a matter-light dual-layer U2, serving as a building block for a multilayer matter-light disinfector according to an embodiment of the present disclosure, and FIG. 5B illustrates its cross-sectional schematic view along the AA′ cut. Matter-light dual-layer U2 as shown is of a cuboid shape, it may also be of other suitable shape, such as cylindrical shape. Matter-light dual-layer U2 includes a matter layer 20 and a light layer 30. Matter layer 20 can be of a flat structure (i.e., with a flat surface), or a non-flat structure with a concave or convex surface, or any other suitable structure as long as the desired filtration purpose can be achieved. Matter layer 20 comprises a porous material 21, a mesh 22 housing the porous material 21, and a frame 15 surrounding the perimeter or sidewall of porous material 21 and mesh 22. As shown in FIGS. 5B and 5C, the space bounded by matter layer(s) 20 defines a light layer 30, which is permeable to fluids and transparent to germicidal light, when in use, is filled with germicidal light (GL). Formed on the upper and lower surfaces of frame 15 are two notches 151, respectively. Notches 151 shown in FIG. 5B are grooves running on the entire upper and lower surfaces, respectively. Frame 15 provides mechanical support for matter layer 20 and confines germicidal light (GL), as well as guides air flow through matter layer 20 and light layer 30. Frame 15 can be made of stainless steel, aluminum alloys, UV resistant plastics, et al, allowing no fluid penetration through its sidewalls. And mesh 22 can also be made of stainless steel, aluminum alloys, UV resistant plastics, et al. Matter-light dual-layers U2 can be used as a building block for MLML disinfector 2, as shown in FIG. 5C. FIG. 5C illustrates a cross-sectional schematic of MLML disinfector 2, made via series connecting a few (4 as shown) matter-light dual-layers U2. Frame notches 151 are to hold O-rings 152, such as rubber O-rings, to provide airtight sealing when connecting a matter-light dual-layer U2 to another, or to an air inlet or outlet channel or duct. As shown in FIG. 5C, a light layer 30 of one matter-light dual-layer U2 and a matter layer 20 of a succeeding matter-light dual-layer U2 including the corresponding frame 15 form a disinfection unit 3020. Matter layer 20 can be a filter with suitable MERV ratings. Also, light layer 30 is a space and, when in use, filled with germicidal light (GL), which in matter-light dual-layer U2 is delivered by a GL source UVC LEDs 351, or those previously described in this specification. Also seen in FIG. 5B, in a matter-light dual-layer U2 UVC LEDs 351 can be physically in contact with matter layer 20, or be positioned with a distance to matter layer 20 to facilitate replaceability of matter layer 20. GL in light layer 30 impinges onto the surface of the matter layer 20 of a succeeding matter-light dual-layer U2, along the fluid flowing direction. In embodiments, UVC LEDs 351 are mounted on printed circuit board (boards) (PCB), which can be mounted on the frame 15. Electrical circuits on the PCB connect the UVC LEDs 351 to external electrical cables.

Worth noting is that MLML disinfector 2 shown in FIG. 5C is constructed via series connecting 4 matter-light dual-layers U2, but only has three effective disinfection units 3020.

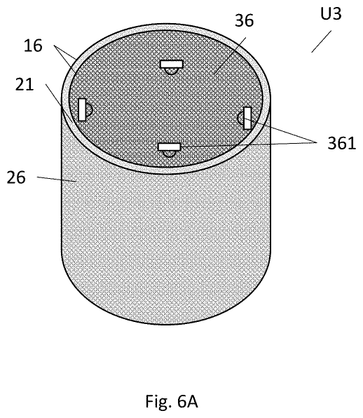

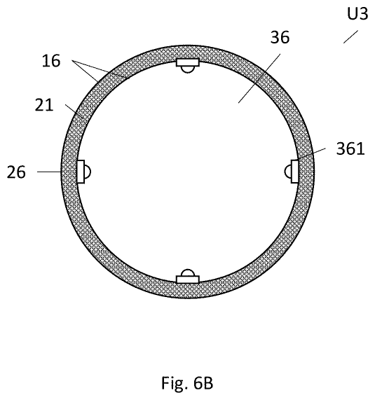

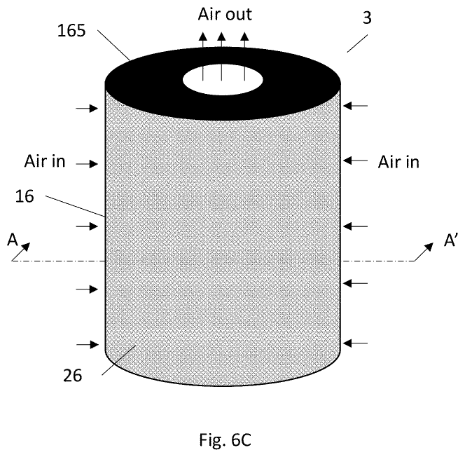

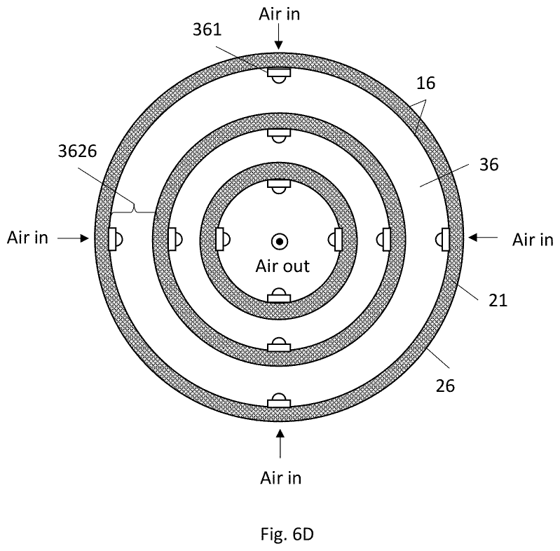

FIG. 6A shows a perspective view of a matter-light dual-layer U3, serving as a building block for another multilayer matter-light disinfector according to another embodiment of the present disclosure, and FIG. 6B illustrates a birds'-eye view of the matter-light dual-layer U3 shown in FIG. 6A. As seen, matter-light dual-layer U3 is shaped as a hollow cylindrical shell, including a cylindrical matter layer 26 (which includes frame 16 and porous material 21) and a light layer 36. As seen from FIGS. 6A, 6B and 6D, light layer 36 is bounded by matter layer 26 and permeable to fluids and transparent to germicidal light, and when in use, is filled with germicidal light. Frame 16 houses porous material 21 and provides mechanical support for matter layer 26. For example, Frame 16 can be a stainless-steel or aluminum alloy mesh housing porous material 21. Light layer 36 is a space, designed to be filled with germicidal light (GL) when in operation, and the GL is delivered by a number of UVC LEDs 361. Also seen in FIGS. 6A and 6B, in matter-light dual-layer U3 UVC LEDs 361 can be physically in contact with matter layer 26, or be displaced with a distance to matter layer 26 to facilitate replaceability of matter layer 26. When UVC LEDs 361 are displaced a distance from matter layer 26, they may obtain mechanical support from frame 16. In some embodiments, UVC LEDs 361 are mounted on a printed circuit board (PCB), which is mounted on the frame 16. Electrical circuits on the PCB connect the UVC LEDs 361 to external electrical cables. Matter-light dual-layers U3 of different diameters (but of the same height) can be used as building blocks for MLML disinfector 3, as shown in FIGS. 6C and 6D. FIG. 6C shows a perspective view of MLML disinfector 3, which can be formed via series connecting a few matter-light dual-layers U3. FIG. 6D illustrates a cross-sectional schematic view of MLML disinfector 3 shown in FIG. 6C along the AA′ cut, revealing that three matter-light dual-layers U3 of different diameters are placed co-axial and connected in series. Seen in FIG. 6D, a light layer 36 of one matter-light dual-layer U3 and a matter layer 26 of a succeeding matter-light dual-layer U3 forms a disinfection unit 3626. GL in light layer 36 impinges onto the surface of the matter layer 26 of a succeeding matter-light dual-layer U3, along the fluid flowing direction. If desired, the very central matter-light dual-layer U3 can also be replaced by a simple matter layer 26.

The two ends of the cylindrical matter-light dual-layers U3 in MLML disinfector 3 are all airtight sealed by a plate 165, to force fluid to enter through the cylindrical side surface of MLML disinfector 3, and flow through the series connected matter-light dual-layers U3, and exit through the central hollow tube formed by the central matter-light dual-layers U3 or a central matter layer. For fluid to enter MLML disinfector 3, frame 16 can be made of a net or mesh structure or other suitable structure to allow the fluid to pass through. For example, frame 16 can be made of stainless-steel or aluminum alloy mesh.

The present disclosure has been described using exemplary embodiments. However, it is to be understood that the scope of the present disclosure is not limited to the disclosed embodiments. On the contrary, it is intended to cover various modifications and similar arrangement or equivalents which can be obtained by a person skilled in the art without creative work or undue experimentation. The scope of the claims, therefore, should be accorded the broadest interpretation so as to encompass all such modifications and similar arrangements and equivalents.

What is claimed is:

1. A fluid disinfector comprising:
one or more disinfection units connected in series along a flow path of a fluid to be disinfected so that the fluid to be disinfected can only enter one disinfection unit after exiting from an adjacent preceding disinfection unit, the disinfection unit comprising a matter layer, a light layer and a germicidal light source for generating germicidal light, wherein:
the matter layer is made of porous material having a first surface to receive the fluid to be disinfected and to intercept and capture pathogens from the fluid to be disinfected;
the light layer is a space permeable to the fluid and transparent to the germicidal light, when in operation, the light layer contains the germicidal light impinging on the first surface of the matter layer.

2. The fluid disinfector according to claim 1, wherein the matter layer comprises a filter selected from a cellulose filter, a polycarbonate filter, a gelatin filter, a polytetrafluoroethylene filter, an activated carbon fiber filter, a granular activated carbon filters, a silica gel filter, an activated alumina filter, a porous or meshed aluminum filter, and a synthetic zeolites filter.

3. The fluid disinfector according to claim 1, wherein a thickness of the matter layer is in the range of 1-10 mm and a thickness of the light layer is in the range of 1-100 cm.

4. The fluid disinfector according to claim 1, wherein the disinfection unit further comprises a frame surrounding a side wall of the matter layer to guide flow of the fluid to be disinfected and, together with the matter layer, defining the light layer; and the frames of the adjacent disinfection units are airtight connected.

5. The fluid disinfector according to claim 4, wherein the germicidal light source is an ultraviolet light emitting diode mounted on the frame.

6. The fluid disinfector according to claim 5, wherein the matter layer is made of porous materials reflecting ultraviolet light.

7. The fluid disinfector according to claim 6, wherein the matter layer is made of materials selecting from porous polytetrafluoroethylene (PTFE), aluminum or meshed aluminum.

8. The fluid disinfector according to claim 1, wherein the matter layer comprises an activated carbon fiber filter of MERV rating in the range of 6-14.

9. A fluid disinfector comprising n disinfection units connected in series such that the fluid disinfector has an effective flow disinfection efficiency ρ_{eff} satisfying:
ρ_{eff}=1−(1−ρ)ⁿ
where ρ is flow disinfection efficiency of a disinfection unit,
$$ρ = 1 - \frac{[ {n}_{o u t} ]}{[ {n}_{i n} ]} , [ {n}_{in} ] , [ {n}_{o u t} ]$$
are pathogen concentrations in the fluid just before and after the disinfection unit, respectively, and n is a positive integer no less than 1, wherein:
the disinfection unit comprising a matter layer, a light layer and a germicidal light source for generating germicidal light, and,
the matter layer is made of porous material having a first surface to receive the fluid and to intercept and capture pathogens from the fluid;
the light layer is a space permeable to the fluid and transparent to the germicidal light, when in operation, the light layer contains the germicidal light impinging on the first surface of the matter layer.

10. The fluid disinfector according to claim 9, wherein n is in the range of 2-6.

11. A fluid disinfector comprising:
one or more cylindrical disinfection units with different diameters co-axially connected in series along a flow path of a fluid to be disinfected so that the fluid to be disinfected can only enter one disinfection unit after exiting from an adjacent preceding disinfection unit, the disinfection unit comprising a matter layer, a light layer, and a germicidal light source for generating germicidal light, wherein:
the matter layer has a cylindrical shape and is made of porous material having a first surface to intercept and capture pathogens from the fluid to be disinfected, and a second surface opposite to the first surface;
the light layer is defined between the first surface of the matter layer of a disinfection unit and the second surface of the matter layer of an adjacent disinfection unit;
when in operation, the germicidal light in the light layer impinges onto the first surface of the matter layer.

12. The fluid disinfector according to claim 11, wherein the matter layer comprises a filter selected from a cellulose filter, a polycarbonate filter, a gelatin filter, a polytetrafluoroethylene filter, an activated carbon fiber filter, a granular activated carbon filters, a silica gel filter, an activated alumina filter, a porous or meshed aluminum filter, and a synthetic zeolites filter.

13. The fluid disinfector according to claim 11, wherein a thickness of the matter layer is in the range of 1-10 mm and a thickness of the light layer is in the range of 1-100 cm.

14. The fluid disinfector according to claim 11, wherein the matter layer contains a frame for mechanical support and the germicidal light source is an ultraviolet light emitting diode mounted on the frame.

15. The fluid disinfector according to claim 14, wherein the matter layer is made of materials selecting from porous polytetrafluoroethylene (PTFE), aluminum or meshed aluminum.

16. The fluid disinfector according to claim 11, wherein two ends of the cylindrical disinfection units are airtight sealed by a plate, respectively.

17. The fluid disinfector according to claim 11, comprising n disinfection units connected in series and having effective flow disinfection efficiency ρ_{eff}:
ρ_{eff}=1−(1−ρ)ⁿ
where ρ is flow disinfection efficiency of a disinfection unit,
$$ρ = 1 - \frac{[ {n}_{o u t} ]}{[ {n}_{i n} ]} , [ {n}_{in} ] , [ {n}_{o u t} ]$$
are pathogen concentrations in the fluid just before and after a disinfection unit, respectively, and n is a positive integer no less than 1.

18. The fluid disinfector according to claim 17, wherein n is in the range of 2-6.

19. The fluid disinfector according to claim 11, wherein the matter layer comprises an activated carbon fiber filter of MERV rating in the range of 6-14.
